(19) United States
(12) Patent Application Publication
(10) Pub. No.: US 2012/0125352 A1
(43) Pub. Date: May 24, 2012

(54) Tobacco Curing Method

(76) Inventor: Jonnie R. Williams, Bradenton, FL (US)

(21) Appl. No.: 13/360,854

(22) Filed: Jan. 30, 2012

Related U.S. Application Data
(62) Division of application No. 12/342,192, filed on Dec. 23, 2008, now Pat. No. 8,151,804.

(51) Int. Cl. A24B 13/00; A61K 36/81; A24F 47/00

(52) U.S. Cl. 131/270; 131/352; 424/751

(57) ABSTRACT

A method of curing tobacco comprises drying a harvested tobacco plant in a controlled environment for a time sufficient to substantially prevent the formation of at least one nitrosamine. The tobacco is first subjected to the controlled environment while at least a majority of the tobacco is in a green state. The resulting cured tobacco usually has tobacco-specific nitrosamine (TSNA) levels which are undetectable and are similar to levels found in freshly harvested, green tobacco.

### CROSS REFERENCE TO RELATED APPLICATION

This is a division of U.S. application Ser. No. 12/342,192, filed Dec. 23, 2008, the disclosure of which is hereby incorporated by reference.

### BACKGROUND

Fresh-cut, green tobacco has virtually no nitrosamine carcinogens. See Wiernik et al., “Effect of Air-Curing on the Chemical Composition of Tobacco,” Recent Advances in Tobacco Science, Vol. 21, pp. 39 et seq., Symposium Proceedings 49th Meeting Tobacco Chemists' Research Conference, Sep. 24-27, 1995, Lexington, Ky. On the other hand, cured tobacco is known to contain a number of nitrosamines, including the harmful carcinogens N′-nitrosonornicotine (NNN) and 4-(N-nitrosomethylamino)-1-(3-pyridyl)-1-butanone (NNK). However, fresh-cut green tobacco is generally considered unsuitable for smoking or other consumption.

Tobacco-specific nitrosamines (TSNAs) are formed primarily during the curing process. It is believed the amount of tobacco-specific nitrosamine (TSNA) in cured tobacco leaf is dependent on the accumulation of nitrites, which accumulate during the death of the plant cell and are formed during curing by the reduction of nitrates under conditions approaching an anaerobic (oxygen deficient) environment. The reduction of nitrates to nitrites occurs by the action of micro flora on the surface of the leaf under anaerobic conditions, and this reduction is particularly pronounced under certain conditions (e.g., humid conditions). During the curing process, the tobacco leaf emits carbon dioxide, which can further dilute oxygen levels in the environment. Once nitrites are formed, these compounds are believed to combine with various tobacco alkaloids, including pyridine-containing compounds, to form nitrosamines.

Williams U.S. Pat. No. 6,202,649, to the present inventor, describes a method of substantially preventing formation of TSNA by, among other things, curing tobacco in a controlled environment having a sufficient airflow to substantially prevent an anaerobic condition around the vicinity of the tobacco leaf. The controlled environment is provided by controlling one or more curing parameters, such as airflow, humidity, and temperature. In practice, Virginia flue tobacco curing according to the method described in Williams '649 typically has a content of N′-nitrosonornicotine (NNN) up to about 0.05 μg/g, a content of 4-(N-nitrosomethylamino)-1-(3-pyridyl)-1-butanone (NNK) up to about 0.05 μg/g, and contents of N′-nitrosoanatabine (NAT) plus N′-nitrosoanabasine (NAB) up to about 0.1 μg/g. Although these TSNA levels are dramatically lower than levels obtained using other curing methods, in some cases it may be desirable to obtain even lower TSNA levels, such as for tobacco used in smokeless products or pharmaceuticals that are orally ingested.

### SUMMARY

In one aspect, a method of substantially preventing the formation of nitrosamines in harvested tobacco comprises drying a tobacco leaf in a controlled environment having a sufficient airflow to substantially prevent an anaerobic condition around the vicinity of the leaf. The controlled environment may be provided by controlling one or more curing parameters, such as airflow, humidity, and temperature. At the time the tobacco leaf is first subjected to the controlled environment, it is in a freshly harvested, green state or at least a majority of the leaf is in a green state. By subjecting tobacco to the controlled environment while in such a state, it is possible to virtually eliminate formation of TSNA during the curing process.

In another aspect, a tobacco product such as cigarettes, cigars, chewing tobacco, snuff, tobacco-containing gum and lozenges, or powdered tobacco-based smokeless tobacco products, is prepared by forming the product from cured tobacco leaf that has been dried in a controlled environment beginning while at least a majority of the tobacco leaf is in an uncured, green state. The cured tobacco or its extract may be used to prepare pharmaceutical products for smoking cessation and/or other therapeutic treatments.

### DETAILED DESCRIPTION

In accordance with the teachings of Williams U.S. Pat. No. 6,202,649, the disclosure of which is hereby incorporated by reference in its entirety, an appropriate combination of parameters such as humidity, rate of temperature change, temperature, time of treatment of the tobacco, airflow, CO level, CO₂ level, O₂ level, and arrangement of the tobacco leaves can be selected to substantially prevent the formation of TSNA during tobacco curing. For a given set of ambient conditions, it may be necessary to adjust, within the curing apparatus or barn, one or more of these parameters. For example, it may be possible to prevent the formation of TSNAs by simply providing a relatively high airflow through the curing barn. In other situations, a lower airflow can be used, provided that other parameters such as humidity, temperature, etc. are appropriately selected.

The practice of tobacco curing is more of an art than a science, as conditions during any given cure must be adjusted to take into account such factors as varietal differences, differences in leaves harvested from various stalk positions, differences among curing barns in terms of where they are used, and environmental variations during a single season or over multiple seasons, especially in terms of weather fluctuations during air-curing. The practice of flue curing is empirical to a certain degree, and is optimally carried out by individuals who have accumulated experience in this art over a significant period of time. See, e.g., Peele et al., “Chemical and Biochemical Changes During The Flue Curing Of Tobacco,” Recent Advances In Tobacco Science, Vol. 21, pp. 81 et seq., Symposium Proceedings 49th Meeting Chemists' Research Conference, Sep. 24-27, 1995, Lexington, Ky. Thus, one of ordinary skill in the art of tobacco curing would understand that the outer parameters described herein, in their broadest forms, are variable to a certain extent depending on the precise confluence of the above factors for any given harvest.

The customary process used for curing green tobacco depends on the type of tobacco harvested. For example, Virginia flue (bright) tobacco is typically flue-cured, whereas Burley and certain dark strains are usually air-cured. The flue-curing of tobacco typically takes place over a period of five to seven days compared to about one to two or more months for air-curing. Flue-curing is generally divided into three stages: yellowing (35-40° C.) for about 36-72 hours (although others report that yellowing begins sooner than 36 hours, e.g., at about 24 hours for certain Virginia flue strains), leaf drying (40-57° C.) for 48 hours, and midrib (stem) drying (57-75° C.) for 48 hours. Many major chemical and biochemical changes begin during the yellowing stage and continue through the early phases of leaf drying.

In a typical flue-curing method, the yellowing stage is carried out in a barn. During this phase the green leaves gradually lose color due to chlorophyll degradation, with the corresponding appearance of the yellow carotenoid pigments. The yellowing stage typically is accomplished by closing external air vents in the barn, and holding the temperature at approximately 100-110° F. for about 3 to 5 days. The yellowed tobacco has a reduced moisture content, e.g., from about 90 wt % when green, versus about 40-70 wt % when yellow. After the yellowing stage, the air vents are opened, and the heat is gradually and incrementally raised to cure the tobacco over a period of about 5 to 7 days. At the conclusion of this period, moisture content in the tobacco usually is about 4-5 wt %. Often the cured tobacco is then subjected to reordering, which increases moisture content to about 11-15 wt %.

The exact mechanism by which tobacco-specific nitrosamines are formed is uncertain, but is believed to be enhanced by microbial activity, involving microbial nitrate reductases in the generation of nitrite during the curing process. TSNAs are believed to be formed upon reaction of amines with nitrite-derived nitrosating species, such as NO₂, N₂O₃ and N₂O₄ under acidic or anaerobic conditions. Tobacco leaves contain an abundance of amines in the form of amino acids, proteins, and alkaloids. The tertiary amine nicotine is the major alkaloid in tobacco, while other nicotine-type alkaloids are the secondary amines nornicotine, anatabine, and anabasine. Tobacco typically contains up to 5% of nitrate and traces of nitrite. TSNA formation is affected by such factors as plant genotype, plant maturity at harvest, curing conditions, and microbial activity.

Nitrosation of nornicotine, anatabine, and anabasine gives the corresponding nitrosamines: N′-nitrosonornicotine (NNN), N′-nitrosoanatabine (NAT), and N′-nitrosoanabasine (NAB). Nitrosation of nicotine in aqueous solution affords a mixture of 4-(N-nitrosomethylamino)-1-(3-pyridyl)-1-butanone (NNK), NNN, and 4-(N-nitrosomethylamino)-4-(3-pyridyl)-1-butanal (NNA). Less commonly encountered TSNAs include NNAL (4-N-nitrosomethylamino)-1-(3-pyridyl)-1-butanol), iso-NNAL (4-N-nitrosomethylamino)-4-(3-pyridyl)-1-butanol) and iso-NNAC (4-(N-nitrosomethylamino)-4-(3-pyridyl)-butanoic acid).

Studies have shown that nitrite and TSNA accumulate during air-curing at the time intervals starting after the end of yellowing and ending when the leaf turns completely brown, e.g., 2-3 weeks after harvest for certain air-cured strains, and approximately a week or so after harvest in flue-cured varieties. This is the time during which loss of cellular integrity occurs, due to moisture loss and leakage of the content of cells into the intercellular spaces. Therefore, there is a short window in time during air-curing when the cells have disintegrated, making the nutrition available for microorganisms. Wiernik et al have suggested that nitrite may then substantially accumulate as a result of dissimilatory nitrate reduction, thus rendering formation of TSNA possible.

There are a few published reports on the effects of microbial flora on the tobacco leaf during growth and curing and on cured tobacco, as cited in Wiernik et al. However, the involvement of microbial nitrite reductases in the generation of nitrate during curing is presumed. When cell structure is broken down after the yellow phase, and nutrients are made accessible to invading microorganisms, these may produce nitrite under favorable conditions, i.e., high humidity, optimal temperature, and anoxia.

As described in Williams '649, a window exists during the tobacco curing cycle in which the tobacco can be treated in a manner that will substantially prevent the formation of TSNA. The precise window during which TSNA formation can be substantially prevented depends on the type of tobacco and a number of other variables, including those mentioned above. Williams '649 describes the window as corresponding to a timeframe post-harvest when the leaf is yellow or undergoing the yellowing process, before the leaf turns brown, and prior to the substantial loss of cellular integrity. During this time frame, the leaves are susceptible to having the formation of TSNAs substantially prevented by subjecting the tobacco to a controlled environment as previously described. This treatment provides a dried, golden yellow leaf suitable for human consumption and, in practice, typically yields an NNN content up to about 0.05 μg/g, an NNK content up to about 0.05 μg/g, and an NAT+NAB content up to about 0.1 μg/g.

It has now been discovered that cured tobacco having levels of TSNAs even lower than those obtained by the method described in Williams '649 may be obtained by subjecting tobacco to a controlled environment while the tobacco is in a freshly-harvested, uncured, green state or shortly after onset of yellowing, e.g., such that at least a majority of the leaf is in the green state. While not wanting to be bound by theory, it is believed that the chlorophyll present in the leaf may block reduction of nitrate to nitrite, which in turn prevents nitrosation of alkaloids into TSNAs as previously described.

In one aspect, prior to subjecting uncured tobacco to a controlled environment as described herein, the yellowing stage is significantly shortened or omitted altogether. Thus, compared to the method described in Williams '649, the tobacco is less ripe at the time at which it is first subjected to the controlled environment. While the timeframe and conditions used for yellowing may vary depending on such factors as tobacco variety, climate, and the like, and further may vary from harvest to harvest and growing season to growing season for reasons previously discussed, the period for yellowing typically ranges from 0 to about 36 hours, more usually from about 18 to about 24 hours. For example, freshly harvested Virginia flue tobacco may be placed in a barn for about 18-24 hours with air recirculation at a temperature of 100-110° F.

In general, when the yellowing stage is omitted or the yellowing period is less than about 12 hours, the tobacco more or less remains in a freshly harvested, green state. As the yellowing period approaches the upper end of the aforementioned range (e.g., 24-36 hours), the relative proportion of yellow increases, e.g., the tobacco approaches a state that no longer has a majority in the green state. In general, yellowing may be carried out to an extent that surface moisture is dried, but without the significant reductions in moisture content associated with conventional yellowing. Usually, the moisture content of the tobacco after the abbreviated yellowing stage ranges from about 55 to about 85 wt %, often from about 65 to about 75 wt %.

In another aspect, in addition to shortening or omitting the yellowing stage, the tobacco may be harvested while it is in a less mature state than the state in which it is normally harvested. Less mature tobacco generally is characterized as having leafs that have smaller size and/or body than those of fully mature leafs. Also, a less mature plant typically has a greater proportion of green color throughout the plant, e.g., the plant is entirely green or only a small fraction of the plant has begun to turn yellow.

The conditions for curing tobacco in a controlled environment that may be used to substantially prevent formation of TSNA are detailed in Williams '649 and will be briefly summarized below. The controlled environment is principally defined by an airflow sufficient to substantially prevent an anaerobic condition around the vicinity of the leaf, and may be created by controlling one or more curing parameters such as airflow, temperature, and humidity. A commercially available dehumidifier or humidifier may be used to control humidity levels. For example, heated or unheated air may be dehumidified air to a relative humidity level of less than about 85%, less than about 60%, or less than about 50% in the curing barn.

The air may be fresh outside air, and should be free or substantially free of combustion exhaust gases. As discussed in Williams '649, combustion exhaust gases, including water vapor, carbon monoxide, and carbon dioxide, dilute ambient oxygen levels, creating anaerobic conditions that lead to TSNA formation through microbial activity. The air may be recirculated as long as an anaerobic condition is substantially prevented.

The temperature within the curing barn typically ranges from ambient (e.g., unheated air) to about 250° F. or more. Excessive temperatures may lead to charring the tobacco and should be avoided. For example, the curing temperature may range from about 100° F. to about 250° F., often from about 160° F. to about 170° F. The optimum temperature within the curing barn can be determined for each case, depending on environmental conditions, tobacco variety, and the like.

The determination of the time for treating the tobacco in the controlled environment may be determined by trial and error. Most often, the treatment time ranges from about 2-4 days. Due to shortening or omitting the yellowing stage, the overall time for processing the tobacco from harvest may be reduced, for example by about 18 to 48 hours, compared to the method described in Williams '649.

The arrangement of the tobacco leaves in the barn is not critical, but it may be advantageous to maximize the exposed surface area of the tobacco leaves. Air circulation within the barn may be of a vertical or horizontal draft design, with the flow of air being in any suitable direction, with manually or automatically controlled fresh air dampers and weighted exhaust dampers. The barn may include a heat exchanger system supplied with a flame detector, igniter wire, sensor cable, dual valve gas train and/or air proving switch.

The resulting cured tobacco typically has individual contents of the nitrosamines NNN, NNK, NAT, and NAB that are below detection limits, e.g., below 0.02 μg/g, as well as a collective content of NNN, NNK, NAT, and NAB that are below detection limits.

The methods described herein may be used with all strains of tobacco, including flue (bright) varieties, Burley varieties, dark varieties, oriental/Turkish varieties, etc. The cured tobacco may be used in any type of tobacco products, non-limiting examples of which include cigarettes, cigars, chewing tobacco, snuff, and tobacco-containing gum, lozenges, and dissolvable strips. The cured tobacco is particularly suitable for use in smokeless products prepared from powdered tobacco, as described in Williams U.S. Pat. Nos. 6,834,654 and 6,668,839, the disclosures of which are hereby incorporated by reference in their entireties. As described in Williams '654 and '839, powdered tobacco-based smokeless products may be prepared from tobacco extracts or from pulverized tobacco. The cured tobacco, typically in extract form, also may be used to prepare pharmaceutical products for smoking cessation and/or other therapeutic treatments. As will be appreciated by persons skilled in the art, because the tobacco is cured while in a less ripe state, some consumers may consider properties such as color and taste less desirable for some types of products such as cigarettes.

### EXAMPLES

The following examples are provided for illustrative purposes only and should not be construed as limiting the scope of the present invention. Examples 1-3 illustrate curing tobacco in a controlled environment beginning while a majority of the tobacco was in a green state. Comparative Examples 1 and 2 illustrate curing tobacco in a controlled environment beginning while a majority of the tobacco was in a yellow state.

Harvested green tobacco was placed in a curing barn at 105° F. with the external air vents closed at an airflow of about 25,000 CFM for yellowing (except for Example 1, where the yellowing stage was omitted). At the conclusion of the yellowing stage, the external air vents were opened and the air temperature was increased to 165° F. for a period of about 2-4 days. Table 1 below indicates the approximate time periods and condition of each tobacco sample at the end of the yellowing stage, and the levels of NNN, NAT, NAB, and NNK measured in the resulting cured tobacco.

TABLE 1

| | % Yellow at end | | | | | | |
|---|---|---|---|---|---|---|---|
| | of yellowing | Yellowing | NNN | NAT | NAB | NNK | TSNA |
| Example | stage | Time (hr) | (μg/g) | (μg/g) | (μg/g) | (μg/g) | (μg/g) |
| 1 | 0 | 0 | N.D. | N.D. | N.D. | N.D. | N.D. |
| 2 | 20 | 18-24 | N.D. | N.D. | N.D. | N.D. | N.D. |
| 3 | 35 | 24-30 | N.D. | N.D. | N.D. | N.D. | N.D. |
| Comp. 1 | 80 | 36-48 | N.D. | 0.067 | N.D. | 0.023 | 0.090 |
| Comp. 2 | 100 | 60-72 | 0.095 | 0.056 | N.D. | N.D. | 0.151 |

N.D. = below detection limit

While particular embodiments of the present invention have been described and illustrated, it should be understood that the invention is not limited thereto since modifications may be made by persons skilled in the art. The present application contemplates any and all modifications that fall within the spirit and scope of the underlying invention disclosed and claimed herein.

I claim:

1. A tobacco product comprising cured tobacco prepared by:
drying tobacco leaf in a controlled environment and for a time sufficient to substantially prevent formation of at least one nitrosamine, wherein the controlled environment comprises an airflow sufficient to substantially prevent an anaerobic condition around the vicinity of the leaf, and wherein the controlled environment is provided by controlling at least one of humidity, temperature, and airflow;
wherein the tobacco leaf is first subjected to the controlled environment while it is uncured and at least a majority of the leaf is in a green state; and
forming a tobacco product comprising the dried tobacco leaf.

2. The tobacco product of claim 1, wherein the tobacco is a Virginia flue variety.

3. The tobacco product of claim 1, wherein the tobacco is a Burley variety.

4. The tobacco product of claim 1, which is a product selected from the group consisting of cigarettes, cigars, chewing tobacco, snuff, and tobacco-containing gum, lozenges, and dissolvable strips.

5. The tobacco product of claim 1, which is a smokeless tobacco product.

6. The tobacco product of claim 5 wherein the smokeless tobacco product comprises powdered tobacco compressed into a solid bit.

7. The tobacco product of claim 6, wherein the powdered tobacco is prepared from a tobacco extract.

8. The tobacco product of claim 6, wherein the powdered tobacco is prepared from pulverized tobacco.

9. The tobacco product of claim 1 which is a pharmaceutical product.

10. The tobacco product of claim 9, wherein the pharmaceutical product is prepared from a tobacco extract.
